(19) United States
(12) Patent Application Publication
(10) Pub. No.: US 2011/0124121 A1
(43) Pub. Date: May 26, 2011

(54) METHODS FOR PREDICTING WEIGHT LOSS SUCCESS

(75) Inventors: John B. DIXON, South Bank (AU); Andrew F. Dixon, East Melbourne (AU); Joseph Raven, Santa Barbara, CA (US)

(73) Assignee: ALLERGAN, INC., Irvine, CA (US)

(21) Appl. No.: 12/899,122

(22) Filed: Oct. 6, 2010

Related U.S. Application Data
(60) Provisional application No. 61/251,764, filed on Oct. 15, 2009.

(51) Int. Cl. G01N 33/53

(52) U.S. Cl. 436/501; 422/430

(57) ABSTRACT

Methods and kits for predicting weight loss success are provided. The methods generally include the steps of selecting a patient or other person undergoing or considering undergoing a weight loss therapy, obtaining a measurement of one or more hormone responses of the person to caloric intake, and subsequently predicting success of a weight loss therapy based on the hormone response.

This application is based, and claims priority under 35 U.S.C. §120 to U.S. Provisional Patent Application No. 61/251,764 filed on Oct. 15, 2009, and which is incorporated herein by reference.

The present invention generally relates to methods for predicting weight loss success, for example, of a gastric banding patient or candidate therefor.

Obesity is a chronic, metabolic state favoring a positive energy balance which results in excessive fat storage. It has highly significant associated medical, psychological, social, physical and economic co-morbidities. As presently understood, it is a multifactorial, genetically-related, and involves heredity, biochemical, hormonal, environmental, behavioral, public health and cultural elements. Morbid obesity, also referred to as severe obesity, typically is associated with a body mass index (BMI), i.e., the ratio of weight in kg to the square of the height in meters, of equal to, or in excess, of 40 kg/m².

Mortality rates for morbidly obese individuals are more than twice as high as those for otherwise similar normal weight individuals. Comorbidities associated with obesity include, for example, high blood pressure, hypertension, hypercholesterolemia, dyslipidemia, Type 2 (non-insulin dependent) diabetes, insulin resistance, glucose intolerance, hyperinsulinemia, coronary heart disease, angina pectoris, congestive heart failure, stroke, gallstones, cholesystitis, cholelithiasis, gastroeophageal reflux disease (GERD), gout, osteoarthritis, respiratory problems such as obstructive sleep apnea and sleep apnea complications of pregnancy, cancer (e.g., endometrial, breast, prostate, and colon cancers), poor female reproductive health (e.g., menstrual irregularities, infertility, irregular ovulation), bladder control problems (e.g., stress incontinence), uric acid nephrolithiasis, psychological disorders (e.g., depression, eating disorders, distorted body image, and low self esteem), metabolic syndrome, sympathetic dysregulation, and inflammatory disease. Therefore, morbid obesity is an extreme health hazard if left untreated.

Bariatric surgical procedures have been developed and are practiced as a means of controlling obesity and obesity related diseases. Laparoscopic gastric banding involves the placement of a band about the upper portion of the stomach to create a stoma which restricts the intake of food. Tubing connects the band to a subcutaneous port where injection of saline allows adjustment of pressure just below the gastro-oesophageal junction. Recent studies suggest that average weight-loss following gastric banding is around 50% to 60% of excess weight, or 20% to 30% of body weight loss at about 8 years after gastric banding.

It may seem somewhat intuitive that restricting the stomach in this manner would naturally lead to a reduction in caloric intake and thus a reduction in weight. However, is should be appreciated that weight loss following gastric band placement is a result of many complex biological processes and responses of the body to the placement of the band, which are not yet fully understood. It has proven to be quite difficult to predict the likelihood of success of any particular preoperative gastric banding patient.

### SUMMARY OF THE INVENTION

Accordingly, novel methods and kits for predicting weight loss success, for example, for a person undergoing or considering undergoing weight loss therapy, for example, gastric banding, are provided.

In an exemplary embodiment, a method of predicting weight loss success in accordance with the invention generally comprises the steps of selecting a patient or other person undergoing or considering undergoing a weight loss therapy, obtaining a measurement of one or more hormone responses of the person to caloric intake, and subsequently predicting success of a weight loss therapy based on the hormone response/responses. Accordingly, the present invention provides a physician with a useful tool for assessing which patients will most likely benefit from gastric banding, and indeed provides a way for a patient to know what to reasonably expect in terms of weight loss success based on a measurable physiological factor or factors.

In one embodiment, the measurement of a hormone response may be obtained by measuring hormone serum concentrations, for example, one or more gut hormone serum concentrations, at fasting and after caloric intake.

For example, a serum concentration of a hormone, for example, a gastrointestinal (gut) hormone, for example, a pancreatic hormone, for example, a pancreatic polypeptide (PP) hormone may be measured both pre-pranidal and post-prandial.

The measurement of other hormones, particularly serum concentrations of other gut hormones, may be also be useful in accordance with the invention as predictors of weight loss success.

In some embodiments, the step of obtaining a measurement of a hormone response comprises sampling at least one endogenous ligand selected from the group consisting of:
1. Pancreatic Polypeptide (PP)
2. Peptide Tyrosine-Tyrosine (PYY)
3. Oxyntomodulin (OXM)
4. Pro-Opiomelanocortin (POMC)
5. Ghrelin
6. Insulin
7. C-Peptide
8. Tumor Necrosis Factor Alpha (TNF-α)
9. Leptin
10. Glucagon-like Peptide 1 (GLP-1)
11. Glucagon
12. Cholecystokinin (CCK)
13. Cocaine-Amphetamine Regulated Transcript (CART)
14. Neuro-peptide Y (NPY)
15. Agouti-related Peptide (AgRP)
16. Alpha/Beta Melanocyte Stimulating Hormone (α/β-MSH)
17. Glial Derived Neurotrophic Factor (GDNF)
18. Melanin Concentrating Hormone (MCH)
19. Orexin_{A/B}
20. T₃
21. T₄
22. Interleukin-6 (IL-6)
23. Corticotrophin Releasing Hormone (CRH)
24. Serotonin
25. Lactate
26. Somatostatin

In certain embodiments, the endogenous ligand is selected from the group consisting of PP, GLP-1, PYY, CCK, insulin and leptin.

In some embodiments of the invention, the step of predicting comprises comparing the hormone response to a scale which indicates a predicted weight loss success, for example, percentage of body weight predicted to be lost if the person were to undergo the weight loss therapy. In a particular embodiment, the scale is compiled from hormone response data taken from an actual human population. By comparing the person's hormone response to the normal response of the population, a physician can more accurately predict the percentage weight loss the person can be expected to lose, for example, within a few months, or up to a year or more following the weight loss procedure.

In another aspect of the invention, a kit for predicting weight loss success is provided. The kit may include means for sample, fix, and evaluate the biological variable, for example, serum concentration of PP, or other biological variable of interest. In some cases, this might include test-tubes or other containers, with a fixative to prevent the degradation of the hormone, a needle to extract the patient's blood, and secure packing in which to place the sample until it is analyzed.

Each and every feature described herein, and each and every combination of two or more of such features, is included within the scope of the present invention provided that the features included in such a combination are not mutually inconsistent.

### DETAILED DESCRIPTION

Methods for predicting weight loss success, for example, for a person undergoing or considering weight loss therapy, for example, gastric banding, are provided.

In a broad aspect of the invention, a method is provided which generally comprises selecting a patient and obtaining data from the patient for the purpose of evaluating a patient's physiological response to caloric intake. The data may be measurement of the change in a hormone serum concentration from a pre-prandial (fasting) hormone serum concentration to a post-prandial (fed) hormone serum concentration. The hormone response, sometimes hereinafter “meal response” is compared to a chart which provides an indication of a prediction of percentage weight loss for the patient after weight loss therapy, for example, a surgical weight loss procedure such as gastric banding. The chart may be derived from data representing a normal meal response of an actual human population sample.

By determining a pre-therapy or pre-operative patient's meal response, a physician can make an informed prediction of percentage weight loss the patient can realistically expect to achieve if the patient undergoes weight loss therapy, for example, a gastric banding or other surgical or non-surgical weight loss procedure or therapy. Similarly, the meal response of a post-operative patient can be used in accordance with the invention, as a predictor of the weight loss success, for example, long term weight loss success, of the patient.

In a specific embodiment, the hormone response data obtained is a PP serum concentration. It is a surprising discovery by the present inventors that individuals exhibiting a low PP meal response tend to achieve higher weight loss after a gastric banding procedure relative to individuals exhibiting a high PP meal response.

More specifically, the step of determining the patient's PP serum concentration includes, for example, the step of measuring a change in the patient's PP serum concentration in response to ingestion of a meal. Techniques for performing such measurements are well known.

One method of the invention includes comparing the person's PP meal response to a PP meal response scale derived from data showing a PP meal response for a population of individuals. By determining where on the scale the subject patient's meal response falls, a prediction is made as to the percentage of weight loss that can be expected for that person.

In one embodiment, the patient's fasting PP serum concentration is measured and recorded. The patient is provided with a standard meal, for example, a 200 calorie meal, and the patient's PP serum concentrations at between about 30 minutes, about 60 minutes, to about 120 minutes following ingestion of the meal are measured and recorded. For example, a value of the serum concentration is represented by mass of the ligand per unit volume of blood sera. This value is then assessed relative to normal ranges of values within the population. The concentrations of PP are measured at each time point after a standard test meal and the area under the concentration curve (AUC) is calculated. Using results under standardized conditions, it can be predicted those people likely to have a better than average weight loss and those people likely to have a poorer response.

### EXAMPLE 1

In the Figure below, the meal response was an average increase in PP concentration of about 50 pg/ml. Those people having a PP concentration below the average went on to have, on average, a higher percentage of weight loss.

Individuals with a Pancreatic polypeptide meal response (AUC) less than average pg/ml in preoperative testing on average lost twice as the percentage weight loss as those with a response>average pg/ml

In this case, the average meal response was 50 pg/ml but this can vary somewhat between series of measurements as expected for any hormone assay.

It is believed that gastric banding reduces pre-prandial hunger and increases postprandial satiety. Several known and potentially many unknown neural and endocrine satiety signals may be responsible for this effect.

### EXAMPLE 2

PP and PYY responses to a standardized meal in a group of patients before gastric banding and a group of patients who have already undergone gastric banding are analyzed to look for a correlation with weight-loss outcome. It is also examined whether PP or PYY concentrations are altered when gastric band pressure is adjusted (an effect known to change satiety).

### Prospective Study

There were 16 obese subjects tested prior to undergoing laparoscopic gastric banding (LAGB) (preoperative group) with their weight-loss results subsequently followed for a mean 48 months (range 18 to 60 months).

### Cross Sectional Study

There were 17 weight-stable subjects who had already undergone LAGB (postoperative group, mean 26 months post-op, range 18 to 36 months). Seventeen controls were BMI matched to the post operative test group (BMI matched control group) to allow assessment of whether hormone serum concentrations were appropriate for body weight or altered by the weight-loss method. There were no statistical differences between the postoperative group and the BMI matched controls.

The preoperative group at the time of testing were of significantly greater weight and BMI than the postoperative and BMI matched control groups in the cross sectional study. The weight-loss already achieved in the postoperative group (cross sectional study) was not significantly different from the weight-loss subsequently achieved in the preoperative group (prospective study) (p=0.09).

### Meal Test Protocol

Participants fasted from 1900 hrs the previous day. Testing occurred while participants were sedentary. Blood samples were taken at 0700, 0900, 1000 and 1100 hrs. A standardized cereal, milk, and banana breakfast (200 kcal) containing 64% carbohydrate, 20.5% protein and 13.5% fat was consumed at 0900 hrs. Weight and height were measured using the same device and operator.

Optimal vs. Reduced Gastric Band Pressure Crossover

LAGB subjects in the cross-sectional study attended two meal tests; one with optimal band pressure and one with reduced pressure. Optimal pressure was defined as the amount of band pressure (saline volume) maintaining current weight. Reduced pressure was defined as optimal pressure less 2 ml of saline or all saline if 1.5 to 2 ml was present. Band adjustments occurred 2 days (mean 49.4 hours) prior testing and were blinded to the subject and investigators. The mean time between paired tests was 4.5 days (range 2 to 14) and no weight difference was found. Participants were asked to guess their band status after adjustment to assess blinding; 13 (76.5%) were undecided, three (17.5%) were correct and one was incorrect (6%).

### Biochemical Assays

Blood samples were mixed with a protease inhibitor, Aprotinin (Trasylol®, Bayer Pharmaceutical), prior to being centrifuged at 4 degree Celsius. Plasma was then stored at −80 degrees Celsius. All samples were assayed in duplicate using sensitive and specific radioimmunoassays for PP and PYY at the Department of Investigative Medicine, Imperial College London, using previously reported techniques. The PYY assay measured both biologically active isoforms PYY₁₋₃₆ and PYY₃₋₃₆.

### Statistical Analysis

The trapezoid rule was used to obtain area under curve (AUC). Biochemical meal response serum concentrations were measured by calculating the area of the postprandial 2 hrAUC that occurred above the 0900 hr fasting serum concentration (i.e. AUC response to meal with each individual's fasting serum concentration taken as the zero point). Demographic, satiety and biochemical data were displayed as mean and standard error. Group differences were compared by independent two-tailed Student's t-test as appropriate. Correlations were assessed using Pearson correlation coefficients. Linear regression analysis was used to control for potential confounders. A P-value<0.05 was considered statistically significant. All analysis was performed using SPSS for Windows version 15 (SPSS Inc, Chicago, Ill.).

### Results

All study groups displayed a 2.5 to 3.0 fold rise in PP serum concentrations post-prandially while no significant PYY meal response was demonstrated (FIG. 1)

### Prospective Study

Within the preoperative group, a lower PP meal response (AUC method) was associated with higher subsequent percentage weight-loss (R=−0.59, p=0.011, FIG. 2A). The correlation remained significant after correcting for age, gender and BMI. The 8 preoperative subjects with a PP meal response<50 pg/ml lost on average twice as much weight as the 8 subjects with a PP meal response>50 pg/ml (weight-loss 29% Vs 14%, p=0.003, FIG. 3). Within the preoperative group no correlation was found between PYY serum concentrations or meal response, and future weight loss (FIG. 2B).

### Cross Sectional Study

Within the postoperative group, higher percentage weight-loss was associated with a lower PP meal response (R=−0.60, p=0.010, FIG. 4A), but there was no association with fasting PP serum concentrations. There were no differences in PP serum concentrations or meal responses between postoperative and BMI matched control groups. Despite a major satiety change, PP serum concentrations and meal response in the postoperative group did not differ between optimal gastric band pressure and reduced gastric band pressure (FIGS. 5A and 5B).

The postoperative group displayed significantly lower PYY 4 hrAUC serum concentrations compared to the BMI matched controls (−27%, p=0.02). Within the postoperative group there was a significant negative correlation between percentage weight-loss and PYY 4 hrAUC serum concentrations (R=−0.67, p=0.003, FIG. 4B). Despite a major satiety change, PYY serum concentrations and meal response in the postoperative group did not alter between optimal gastric band pressure and reduced gastric band pressure (FIG. 5C).

### Discussion

This study suggests that low PP response to a meal predicts higher weight-loss after gastric banding. It was not only observed that a low PP meal response in preoperative testing predicted greater subsequent weight-loss, but also found it to be strongly related to percentage weight-loss in those who had already undergone LAGB. Furthermore, PP concentrations and response appear unaltered with LAGB weight-loss.

Although hormones such as ghrelin and PYY have been suggested to be related to the success of RYGB, to the inventors' knowledge no gut hormones have actually been shown to predict sustained weight-loss outcomes. This may represent a first time a gut hormone has been found to predict sustained weight loss response to bariatric surgery.

### EXAMPLE 3

A prospective study is used to assess plasma PP and PYY meal responses in 16 obese individuals prior to gastric banding (LAGB). The study examines 17 postoperative individuals who had already achieved mean 28% LAGB-induced weight-loss (range 10% to 38%).

TABLE 1
Demographic and weight characteristics for the study subjects.
Mean (standard error). Significant and near significant
differences (unpaired student t-test) are displayed.

| | Preoperative | | BMI Matched | | Postoperative | | |
|---|---|---|---|---|---|---|---|
| | Test Group | | Controls | | Test Group | | |
| | (n = 16) | | (n = 17) | | (n = 17) | | P-Value |
| Sex (M:F) | 3:13 | | 5:12 | | 3:14 | | |
| Age (years) | 41.1 | (2.1) | 42.9 | (2.4) | 44.5 | (2.3) | |
| BMI (kg/m²) | 42.6 | (2.5)*^{#} | 32.9 | (1.6)^{#} | 31.9 | (1.2)* | Both <0.001 |
| Height (m) | 1.68 | (0.03) | 1.70 | (0.03) | 1.67 | (0.02) | |
| Weight (kg) | 120.5 | (8.4)*^{#} | 95.3 | (4.9)^{#} | 90.3 | (5.0)* | Both <0.01 |
| Postoperative BMI | 33.2 | (2.5) | | | | | |
| (kg/m²) | | | | | | | |
| Postoperative Weight | 93.7 | (5.3) | | | | | |
| (kg) | | | | | | | |
| Preoperative BMI | | | | | 44.2 | (1.7) | |
| (kg/m²) | | | | | | | |
| Preoperative Weight | | | | | 125.4 | (7.0) | |
| (kg) | | | | | | | |
| Weight-loss (kg) | 34.7 | (5.5) | | | 35.1 | (3.1) | |
| Weight-loss (%) | 21.8 | (2.9)* | | | 28.0 | (1.8)* | 0.09 |

| | | |
|---|---|---|
| range | 6 to 47 | 10 to 38 |

### Results

In the prospective study, subsequent mean weight-loss was 22% (range 6% to 47%) after mean 48 months after LAGB. Low preoperative PP meal response (2 hrAUC) predicted significantly higher subsequent weight-loss after LAGB (R=0.59, p=0.011). Individuals with <50 pg/ml PP rise lhr after the meal lost twice as much weight as those with >50 pg/ml PP rise (29% vs. 14%, p=0.003). In the cross-sectional study, individuals with higher weight-loss had lower PP meal responses (2 hrAUC, R=0.60, p=0.011) and lower PYY serum concentrations (4 hrAUC, R=0.67, p=0.003). Comparing studies, PP responses were equivalent between preoperative and postoperative LAGB groups, while postoperative group PYY serum concentrations (4 hrAUC) were reduced compared to the preoperative group (−22% p=0.04) and BMI matched controls (−27% p=0.02).

### Discussion

This study shows that Peptide YY serum concentrations appear reduced following LAGB, perhaps representing attempted orexigenic compensation. PP response to a meal appears to be a predictor of weight loss after gastric banding. Although not wishing to be bound by any particular theory of operation, the reduced PP response may be a sign of reduced vagal nerve response, and one or the other may be an important determinant of individual susceptibility to LAGB weight-loss.

### EXAMPLE 4

A 44 year old female patient explains to her doctor that she has tried to lose weight using diet and exercise but can not seem to keep the weight off. She is considered clinically obese at a height of 5′4″ and weight of 210 pounds. Her medical history reveals no known comorbidities other than slightly elevated blood pressure. The doctor believes she is generally considered a good candidate for laparoscopic adjustable gastric banding (LAGB). Before committing to the surgery, the patient naturally feels the need to find out from the doctor how much weight she might be expected to lose as a result of the surgery.

The doctor explains to the patient that there is a simple procedure he can perform that will result in a scientifically based prediction of her weight loss success after gastric banding surgery. The patient indicates that she would like to have the weight loss success procedure performed as soon as possible.

The patient is told to fast overnight and return to the doctor's office the next morning for a series of simple blood tests.

The patient fasts overnight and returns to the doctor's office at 8:00 the next morning.

A “fasting” blood sample is drawn from the patient.

At 8:15 am, the patient consumes a 300 calorie meal comprising 4 ounces of dehydrated apple, 8 ounces of whole grain cereal and 8 ounces of whole milk.

At 8:45 am, a first postprandial blood sample is drawn.

At 9:15 am, a second postprandial blood sample is drawn.

At 10:15 am, a third postprandial blood sample is drawn.

The patient leaves the office and is told her results will be provided and discussed with her at her next office visit.

The blood samples are sent to a lab and assayed for PP serum concentrations, and more specifically, for the patient's PP meal response. The PP meal response measured generally indicates the difference in PP serum concentration of the patient between her fasting PP serum concentration and her peak postprandial PP serum concentration.

The doctor obtains the PP meal response data from the lab along with a chart indicating the patient's PP serum concentration and the percentage of excess weight loss that can be expected for that patient. The chart is based on PP meal response data from a significant population of patients who had blood samples assayed using identical criteria assays been provided with an identical 300 calorie meal, during the same time of day.

The doctor shares the results of the test with the patient and explains to the patient that she can be expected to achieve up to about 45% excess weight loss four months after weight loss surgery.

In another aspect of the invention, a kit for predicting weight loss success is provided. The kit may include means for sample, fix, and/or evaluate the biological variable, for example, serum concentration of PP, or other biological variable of interest. In some cases, this may include test-tubes or other containers, with a fixative to prevent the degradation of the hormone, a needle to extract the patient's blood, and secure packing in which to place the sample until it is analyzed. For example, the kit may comprise a needle assembly for extracting a sample, for example a blood sample, from a patient, and an assay for determining, from the sample, a serum concentration of a hormone. The kit further includes an evaluation chart to assist a physician in determining a percentage of weight loss to be expected based on the determination.

As described elsewhere herein, the evaluation chart may include data derived from a hormone serum concentration response of an actual human population.

### PP

PP is produced by F-type cells which constitute about 10% of the endocrine pancreas. PP is composed of 36 amino acids, C-terminal amidation is required for bio-activity, and is released bi-modally in response to caloric content of a meal and the degree of gastric distention (probably cholecystokinin mediated).

As the primary member of the PP family of peptides, PP shares a common stereochemistry with neuropeptide Y (NPY) and peptide-tyrosine-tyrosine (PYY). These peptides contain the PP-fold, which is a poly-proline helix and an α-helix which is connected by a β-turn. The PP fold gives pancreatic polypeptide its stereotypical U shape.

PP preferentially binds to Y₄ receptors (Y₄R) and has some low affinity interaction with Y₅ receptors (Y₅R) as well. Several primary physiological responses occur following the activation of Y₄Rs by PP:
a. PP reduces food intake (in humans and mice).
b. PP decreases gastric emptying (in mice).
c. PP reduces weight gain (in humans and mice).

The pharmacological effects of PP in vivo are dependant on its method of administration, however. For example, while peripheral administration of PP induces the anorexogenic (loss of appetite) response, central administration of PP induces orexigenic (hunger) responses. Several studies demonstrated peripheral administration of physiological doses of PP, in mice, produce rapid and prolonged decreases in food intake; acting within 20 minutes and lasting for 24 hours.

Of particular relevance for the utilization of PP as a marker for gastric banding success, concentrations of endogenous PP appear to be reduced in obesity.

Furthermore, while PP has been shown to reduce food intake in normal weight humans further studies need to be conducted to unequivocally determine its effect in the obese. PP has been considered an excellent candidate for treating obesity, as it has a prolonged effect, inhibits ghrelin expression, and is best if administered in the periphery.

Hormones other than PP may also be candidates for utilization as predictive markers for successful response to gastric banding. Included below are several candidates that may also serve to predict weight loss outcome. The list is not considered to be exhaustive however.

Basal serum ghrelin concentrations may be useful as a predictive factor for success. Ghrelin acts to positively reinforce PP expression. Furthermore, ghrelin is a potent activator for growth hormone release. Thus, individuals who exhibit elevated PP concentrations may also be prone to greater anabolic stimuli and greater hunger, thereby predisposing them to a lower likelihood of success for gastric banding therapy.
a. Post-prandial serum cholecystokinin (CCK) response may be useful as a predictive factor for success. CCK acts to increase insulin expression as well as stimulate PP release. Thus, elevations in post-prandial CCK serum concentrations would have the dual role of increasing PP and insulin. The elevations in insulin may then act as an anabolic factor, increasing adiposity and storage of glucose in the form of fatty acids, and ultimately worsening the patients' metabolic state.
b. Both motilin and secretin act to up-regulate PP expression in vivo. As such these factors may also be considered as possible predictive cues and precursors associated with the PP response demonstrated by Dr. Dixon.

Finally, serum concentrations of somatostatin may also serve to predict weight loss outcome, as it is normally a potent inhibitor of PP release.

Unlike current dietary and pharmacological methods of weight loss, modern bariatric surgery has been shown to deliver significant, sustainable weight loss. The success of weight loss surgery implies an ability to influence and override normal energy homeostasis. It has been reported that PYY responses after a meal are increased following Roux-en-Y Gastric Bypass (RYGB) and that this may contribute to the long-term weight loss sustainability of the procedure.

Finding predictors of banding outcomes has proved elusive. The significant variation in weight loss outcome response to LAGB therapy is not well understood. The present invention provides a novel, easy to practice approach to predicting weight loss success for a gastric banding patient or a potential gastric banding patient.

Although the invention has been described and illustrated with a certain degree of particularity, it is understood that the present disclosure has been made only by way of example, and that numerous changes in the combination and arrangement of elements, steps and parts can be resorted to by those skilled in the art without departing from the scope of the invention, as hereinafter claimed.

What is claimed is:

1. A method for predicting weight loss success for a person considering a weight loss therapy, the method comprising the steps of:
selecting a patient or other person undergoing or considering undergoing a weight loss therapy;
obtaining a measurement of a hormone response of the person to caloric intake; and
predicting success of a weight loss therapy based on the hormone response.

2. The method of claim 1 wherein the hormone response is a gut hormone response.

3. The method of claim 1 wherein the step of obtaining a measurement comprises measuring a gut hormone serum concentration.

4. The method of claim 3 wherein the step of determining comprises measuring a gut hormone serum concentration at fasting and after caloric intake.

5. The method of claim 1 wherein the step of obtaining a measurement comprises measuring a pancreatic hormone.

6. The method of claim 1 wherein the step of obtaining a measurement comprises measuring a pancreatic polypeptide (PP) hormone.

7. The method of claim 1 wherein the step of obtaining a measurement comprises measuring a plasma pancreatic polypeptide serum concentration.

8. The method of claim 1 wherein the step of obtaining a measurement comprises measuring a serum concentration of at least one hormone selected from the group consisting of: PP, GLP-1, PYY, CCK, insulin and leptin.

9. The method of claim 1 wherein the step of obtaining a measurement includes measuring a change in the person's pancreatic polypeptide (PP) serum concentration in response to caloric intake.

10. The method of claim 1 wherein the step of predicting comprises comparing the hormone response to a scale.

11. The method of claim 1 wherein the step of predicting comprises comparing the hormone response to hormone response data from an actual population of people.

12. The method of claim 1 wherein the step of predicting comprises comparing the hormone response to a scale including hormone response data from an actual population of people.

13. The method of claim 1 wherein the hormone response is a pancreatic polypeptide (PP) response and the step of determining comprises measuring a PP fasting serum concentration and measuring a PP serum concentration after intake of food.

14. The method of claim 13 further comprising comparing the PP meal response obtained to a scale.

15. The method of claim 13 wherein the step of predicting comprises comparing the PP response to PP response data from an actual population of people.

16. The method of claim 1 wherein the step of determining comprises determining a hormone serum concentration at about 30 minutes to about 60 minutes following caloric intake.

17. The method of claim 16 wherein the caloric intake is at least about 200 calories.

18. A method for predicting weight loss success for a person considering a surgical weight loss procedure or a surgical weight loss patient, the method comprising the steps of:
selecting a patient or other person undergoing or considering undergoing a surgical weight loss procedure;
obtaining measurements of the person's pancreatic polypeptide (PP) serum concentration at fasting and after caloric intake to obtain a PP response for the person;
comparing the person's PP response to data derived from PP responses of an actual population of people; and
predicting weight loss success for the person based on the comparison.

19. The method of claim 18 wherein the step of predicting includes predicting a percentage excess body fat weight loss.

20. The method of claim 18 wherein the step of obtaining includes providing the caloric intake to the person.

21. The method of claim 20 wherein the caloric intake is at least about 200 calories.

22. A kit useful for assisting in predicting weight loss success of a potential gastric banding patient, the kit comprising:
a needle assembly for extracting a sample from a patient;
an assay for determining, from the sample, a serum concentration of a hormone;
an evaluation chart to assist in determining a percentage of weight loss to be expected based on the analysis.

23. The kit of claim 22 wherein the assay is useful for determining a PP serum concentration from a blood sample.

24. The kit of claim 22 wherein the evaluation chart includes data derived from an actual human population.

25. The kit of claim 22 wherein the evaluation chart includes data derived from a hormone serum concentration response of an actual human population.
